(19) United States
(12) Patent Application Publication
(10) Pub. No.: US 2018/0243175 A1
(43) Pub. Date: Aug. 30, 2018

(54) ARGININE-CONTAINING RESTORATIVE DENTAL MATERIALS AND METHODS OF PREVENTING AND CONTROLLING CARIES ASSOCIATED WITH DENTAL WORK

(71) Applicant: THE UNIVERSITY OF FLORIDA RESEARCH FOUNDATION, INC., Gainesville, FL (US)

(72) Inventors: Marcelle Matos Nascimento, Gainesville, FL (US); Saulo Geraldeli, Gainesville, FL (US); Christopher D. Batich, Gainesville, FL (US)

(21) Appl. No.: 15/758,953

(22) PCT Filed: Sep. 7, 2016
(86) PCT No.: PCT/US2016/050520

§ 371 Date: Mar. 9, 2018

Related U.S. Application Data
(60) Provisional application No. 62/216,442, filed on Sep. 10, 2015.

(51) Int. Cl. A61K 6/00; A61P 1/02; A61Q 11/00; A61K 6/083

(52) U.S. Cl. CPC A61K 6/0067; A61K 6/0091; A61K 6/083; A61Q 11/00; A61P 1/02

(57) ABSTRACT

The present disclosure provides arginine-releasing restorative dental materials, methods of making the dental materials, and methods of using arginine-releasing restorative dental materials to and/or to treat, prevent and/or control caries, such as caries associated with dental work.

### CROSS-REFERENCE TO RELATED APPLICATIONS

This application claims the benefit of and priority to U.S. Provisional Application Ser. No. 62/216,442, having the title “ARGININE-CONTAINING RESTORATIVE DENTAL MATERIALS AND METHODS OF PREVENTING AND CONTROLLING CARIES ASSOCIATED WITH DENTAL WORK,” filed on Sep. 10, 2015, the disclosure of which is incorporated herein in by reference in its entirety.

### BACKGROUND

Dental caries is the most prevalent infectious and chronic disease affecting humans, and is associated with costly treatment worldwide. The transition from dental health to dental caries is characterized by compositional and metabolic changes in the complex microbial communities of oral biofilms. Oral biofilms, often called dental plaque, constantly form and grow on all tooth surfaces. Secondary caries can form at the margins of composite restorations. This has been attributed to adhesive failure and consequent accumulation of cariogenic biofilms at the tooth-composite interface. Secondary caries remains the main reason for failure of dental composite restorations, and replacement of the failed restorations accounts for up to 75% of the operative work. The inherent biodegradation of the bond between the tooth and the adhesive layer produces crevices that are readily colonized by caries pathogens such as Streptococcus mutans. Those crevices are also derived from polymerization shrinkage and improper resin-based composite layering. In the microenvironment of a crevice, oral biofilms are protected from fluid flow and salivary buffering, which favors the continuous acid production by S. mutans leading to tooth demineralization.

### SUMMARY

Briefly described, embodiments of the present disclosure provide arginine-containing restorative dental materials that release arginine into the oral cavity of a host, arginine-containing dental adhesive compositions and materials for use in restorative dentistry, methods of reducing caries associated with restorative dental materials by using arginine containing/releasing restorative dental materials, and methods of making the arginine containing/releasing dental compositions and materials.

Embodiments of arginine-containing/releasing dental materials of the present disclosure include restorative dental materials including a restorative dental composition and arginine incorporated into the restorative dental material such that, in the oral cavity of a host, arginine is released from the restorative dental material over time.

Embodiments of dental adhesive compositions of the present disclosure for use in restorative dentistry include a polymerizable compound or mixture of compounds capable of polymerizing to form an adhesive bond, an optional filler material, and arginine, where the arginine is released from the dental adhesive composition into an oral cavity of a host over time.

In embodiments, methods of reducing caries associated with restorative dental materials in a host include the steps of providing a restorative dental material including arginine, and placing the restorative dental material in the oral cavity of a host, where the arginine is released from the restorative dental material into the oral cavity of the host over time.

Embodiments of methods of making an arginine-containing restorative dental material of the present disclosure having anti-caries activity includes at least the following steps: providing a restorative dental composition or the ingredients for a restorative dental composition; combining arginine with the restorative dental composition or the ingredients for a restorative dental composition to produce an arginine-containing restorative dental composition; forming the arginine-containing restorative dental material from the arginine-containing restorative dental composition, such that the arginine-containing restorative dental material releases arginine into an oral cavity of a host over time.

Other methods, compositions, features, and advantages of the present disclosure will be or become apparent to one with skill in the art upon examination of the following drawings and detailed description. It is intended that all such additional compositions, methods, features, and advantages be included within this description, and be within the scope of the present disclosure.

### BRIEF DESCRIPTION OF THE DRAWINGS

Further aspects of the present disclosure will be more readily appreciated upon review of the detailed description of its various embodiments, described below, when taken in conjunction with the accompanying drawings, described in the Examples below.

FIG. 1 illustrates phase separation occurring when HDDMA was added last to an embodiment of an arginine-containing dental adhesive composition of the present disclosure.

FIG. 2 demonstrates a bar-shaped specimen of an embodiment of arginine-containing dental adhesive of the present disclosure prior to load application in a three point bending flexural test.

FIG. 3 is a photograph of a sound third molar tooth sample sectioned in 4 quarters. Each quarter was randomly used to accommodate one of the arginine-containing dental adhesive concentrations (control (0%), 5%, 7% and 10%) of the present disclosure to be tested.

FIG. 4 is a photograph of color-identified dentin quarters sectioned with diamond saw disc to obtain beams for microtensile test. Colors are not shown in the black and white image, so location of colors is provided here for reference: top left quarter: blue, top right quarter: green, lower left quarter: red, lower right quarter: black.

FIG. 5 is a photograph of a single glued beam from FIG. 4 as placed in the Geraldeli-V2 device prior to application of the load in the microtensile bond strength test machine.

FIG. 6A-6D are SEM images of the resin-dentin interface created with an embodiment of the adhesive systems of the present disclosure containing arginine in different concentrations: FIG. 6A: Arg0 (control), FIG. 6B: Arg5, FIG. 6C: Arg7 and FIG. 6D: Arg10. C=composite; AD=adhesive layer; D=dentin; white arrows indicates different sizes of arginine particles within the adhesive layer. Magnification used was approximately from 1.140 to 1.510×.

FIGS. 7A and 7B are graphics showing the release rates of arginine from the testing bonding agent containing 7% arginine over time.

FIG. 8 is a bar graph showing cumulative arginine release over time from the testing bonding agent with 7% arginine.

FIG. 9 is a bar graph showing cumulative arginine release from the testing bonding agent with 7% arginine per cycle over 3 days or arginine recharge.

FIGS. 10A and 10B are graphs showing planktonic growth curves of tested bacterial strains in the presence of adhesives. +A: growth in the presence of Arg7 discs; −A: growth in the presence of Arg0 discs; C: control, growth in the absence of adhesive discs.

FIG. 11 shows digital images of biofilm formation in glass slides and by confocal microscopy. Blank squares in the first column indicate that no biofilm formation by UA159 was observed during in BHI at pH 7.0 in the presence of the adhesive discs.

### DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

Any publications and patents cited in this specification that are incorporated by reference are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of dentistry, biomaterials, biology, medicine, microbiology, chemistry, biochemistry, molecular biology, pharmacology, and the like, which are within the skill of the art. Such techniques are explained fully in the literature.

It must be noted that, as used in the specification and the appended embodiments, the singular forms “a,” “an,” and “the” include plural referents unless the context clearly dictates otherwise. Thus, for example, reference to “a cell” includes a plurality of cells. In this specification and in the embodiments that follow, reference will be made to a number of terms that shall be defined to have the following meanings unless a contrary intention is apparent.

Prior to describing the various embodiments, the following definitions are provided and should be used unless otherwise indicated.

### Definitions

In describing the disclosed subject matter, the following terminology will be used in accordance with the definitions set forth below.

As used in the present application, the term “restorative dental materials” refers to materials used in a host in the field of restorative, preventative, and aesthetic dentistry and includes not only traditional restorative materials (including biomimetic restorative dental materials) but also preventative and aesthetic dental materials. The term “restorative dental materials” is thus used in the present disclosure for purposes of brevity but should not be limited merely to materials strictly for use in restorative dental practice. Thus, the term “restorative dental materials” includes materials such as, but not limited to, dental adhesives (provides bond between tooth tissues and different types of dental materials, such as fillings, prosthodontics, orthodontics, etc.), resin cements (e.g., for fitting crowns and other dental hardware), resin composites, tooth-colored restorative material, materials used for dental fillings (such as, but not limited to, resin modified glass ionomers, endodontic materials), prosthodontics devices such as complete and partial dentures, dental bleaching devices and/or agents, materials used for dental sealants and tooth varnish, and the like. In embodiments of the present disclosure, preventive and restorative dental materials can include permanent (e.g., intended to remain indefinitely in the oral cavity of a host, even if eventual replacement is required) and semi-permanent (e.g., intended to remain in the oral cavity of a host for a specified or estimated length of time, with expectation of eventual removal or replacement) restorative materials (such as, but not limited to, fillings, sealants, varnishes, bonding materials, prosthodontics (e.g., crowns, bridges, complete and partial dentures and other dental implants), as well as materials and devices, such as but not limited to adhesives or other polymers/polymerizable compounds and/or bonding agents used to affix some of the above mentioned preventive and restorative dental materials and implements into the mouth/oral cavity of a host. As used in the present disclosure, “restorative dental materials” do not include temporary oral hygiene compositions such as toothpaste, oral rinses, gels, and the like.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying, slowing, or arresting disease progression, substantially preventing spread of disease, controlling, reducing, amelioration or palliation of the disease state, and remission (partial or total) whether detectable or undetectable. In addition, “treat”, “treating”, and “treatment” can also be therapeutic in terms of a partial or complete cure for a disease and/or adverse effect attributable to the disease. With respect to caries, “treating” includes reducing the appearance of dental caries lesions and slowing or arresting the progression of dental caries lesions (e.g., slowing or stopping the growth or severity of the caries lesions) and restoring or replacing the tooth tissues lost by the caries process. “Treating” also includes “preventing”/“prophylactically treating.” As used herein, the terms “prevent”, “prophylactically treat,” or “prophylactically treating” refers to completely, substantially, or partially preventing a disease/condition or one or more symptoms thereof in a host. Similarly, “delaying the onset of a condition” can also be included in “prophylactically treating”, and refers to the act of increasing the time before the actual onset of a condition in a patient that is predisposed to the condition. With respect to caries, “preventing” or “prophylactic treatment” can include preventing the development and appearance of new caries lesions in a host.

By “administration” is meant introducing a compound of the present disclosure into a subject; it may also refer to the act of providing a composition of the present disclosure to a subject (e.g., by prescribing). The preferred route of administration of the compositions of the present disclosure is oral. However, any route of administration that will assist the composition to treat the oral condition of the host can be used.

The term “organism,” “subject,” or “host” refers to any living entity in need of treatment, including humans, mammals (e.g., cats, dogs, horses, chicken, pigs, hogs, cows, and other cattle), and other living species that are in need of treatment. In particular, the terms “host”, “subject”, and “organism” include humans. As used herein, the term “human host” or “human subject” is generally used to refer to human hosts. In the present disclosure the term “host” typically refers to a human host, so when used alone in the present disclosure, the word “host” refers to a human host unless the context clearly indicates the intent to indicate a non-human host. Hosts that are “predisposed to” condition(s) can be defined as hosts that do not exhibit overt symptoms of one or more of these conditions but that are genetically, physiologically, or otherwise at risk of developing one or more of these conditions (e.g., caries).

As used in the present disclosure, the terms “release” and “recharge” with respect to arginine incorporated in the material of the present disclosure refers to the giving off (“release”) or taking up (“recharge”) of arginine by arginine-containing restorative dental materials/devices or a compound/composition that forms a part of a preventive and restorative dental material. In embodiments, “recharge” refers to a secondary or subsequent taking up of arginine by devices and restorative dental material, as distinguished from the initial incorporation of arginine into the restorative dental material.

As used herein, the following terms have the meanings ascribed to them unless specified otherwise. In this disclosure, “consisting essentially of” or “consists essentially” or the like, when applied to methods and compositions encompassed by the present disclosure, refers to compositions like those disclosed herein but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

### DISCUSSION

The embodiments of the present disclosure encompass restorative dental materials (such as, but not limited to permanent and/or semi-permanent dental compositions and/or materials, prosthodontics devices, and bleaching devices (trays), etc.) containing arginine and capable of releasing arginine into the oral cavity of a host into which the restorative dental material is placed. The present disclosure also provides embodiments of methods of making the restorative dental compositions and materials of the present disclosure as well as methods of using them to prevent and reduce caries in a host, including, but not limited to, new caries and existing caries associated with or located adjacent to the presence of restorative dental materials in a host as well as caries associated with techniques aiming teeth whitening.

Although production of acid by the bacteria in oral biofilms is a direct cause of dental caries, increases in the proportions of aciduric organisms appear to occur at the expense of species that are less acid tolerant (i.e., less “aciduric”). A subset of less aciduric organisms derives protection from plaque acidification by alkali generation, which shows a positive association with dental health. One of the primary routes for alkali generation by oral bacteria is the arginine deiminase system (ADS), through which arginine is catabolized into ornithine, ammonia and CO₂, with the concomitant generation of ATP. Arginine is an amino acid found in a variety of foods, and is also naturally produced by the human body and secreted in saliva in free form or as salivary peptides. Arginine entering the mouth can be metabolized by certain oral bacteria via the ADS to produce ammonia, which neutralizes glycolytic acids and contributes to the pH rise of oral biofilms.

Ammonia production via ADS prompts a neutral environmental pH that is less favorable to the outgrowth of acid-producing cariogenic bacteria, thus reducing caries risk. Hence, the ADS serves key physiological functions in bacteria, providing protection from the deleterious effects of low pH and ATP for growth and maintenance. The ADS activity in oral biofilms can impact the ecology of oral microbial communities by moderating the pH through ammonia production. A variety of bacteria that colonize the teeth and oral soft tissues and form oral biofilms express the ADS. An increased risk for dental caries has been associated with a reduced ability of oral biofilms to produce alkali from arginine via the ADS.

Evidence continues to accumulate from in vitro and clinical observations that support the role of alkali generation in oral ecology and inhibition of dental caries (Dawes and Dibdin, 2001; Margolis et al., 1988b; Nascimento et al., 2009a; Peterson et al., 1985; Shu et al., 2007a; Wjeyeweera and Kleinberg, 1989a). A positive correlation between oral arginine metabolism and absence of caries activity has been clinically demonstrated in adults (Nascimento et al., 2009a), and in children (Nascimento et al., 2013). Such “arginolytic” bacteria, when found in a host (particularly the oral cavity of a host) can be beneficial in increasing ammonia production in the oral cavity of a host, thereby providing an environmental factor for reducing the incidence of caries. Specifically, it has been found that oral bacteria from dental plaque of caries-free subjects present higher ADS activity compared with those from caries-active subjects. There is also a high degree of variability in the rate of ammonia production among individuals, in some cases greater than 1000-fold. It is also possible that environmental conditions and host factors, such as the presence of arginine, encourage differential expression of the ADS in caries-active versus caries-free subjects.

Caries can result from the overgrowth of certain bacteria in the dental plaque on tooth surfaces in the oral cavity of a host. Such dental plaques or oral biofilms can be especially difficult to detect, remove, and/or treat in crevices and other hard-to-reach areas, such as at the margins of tooth restorations and other areas surrounding restorative dental crowns and implants and materials. For instance, biodegradation of the bond between the tooth and the “filling” (e.g., the man made resin interface between tooth structures and adhesive material) bonding material, adhesive layer, etc.) or other restorative dental materials can produce crevices that are readily colonized by caries pathogens such as Streptococcus mutans and Lactobacillus sp. In the microenvironment of a crevice, oral biofilms are protected from fluid flow and salivary buffering, which favors the continuous acid production by S. mutans leading to loss of tooth minerals (demineralization).

As discussed above, when these biofilms occur at the margins or beneath a restorative dental filling or other restorative tooth repair material, it can result in secondary caries or caries adjacent to restorations (CARs). Thus, such secondary caries often results from adhesive or bonding failure and consequent accumulation of cariogenic biofilms at the tooth-composite interface. Secondary caries is a primary cause of restoration failure and necessary replacement of dental composite restorations, and, if not detected early, secondary caries can often result in the need for more invasive and expensive dental restorations and/or surgical procedures, such as root canal.

Although it may not be possible to entirely eliminate biofilms from the crevices, the engineering of novel restorative dental materials, such as dental adhesives, that can shift the microbial ecology from a disease to a health state are greatly desirable. Thus, in the present disclosure, materials and methods are provided for increasing the arginine in an area of a host's oral cavity around restorative dental materials. Providing new methods for increasing ADS activity leading to prevention of caries activity in the area around restorative dental materials could reduce the incidence of secondary caries associated with restorative dental materials and therefore reduce the necessity for replacement and/or additional surgical procedures resulting from the secondary caries.

The discussion and examples below demonstrate the production of a type of restorative dental material (a dental adhesive used in filling materials) into which arginine has been incorporated and a demonstration that addition of arginine does not adversely affect the physical and chemical properties of the material for which it is intended (e.g., bond strength, flexibility, etc.) and that arginine is in fact released from the material over time. The release of arginine from the restorative dental materials of the present disclosure provides a localized source of arginine for ammonia production via ADS of arginolytic bacteria present in oral biofilms of the host, particularly oral biofilms near or adjacent to the site of the restorative dental materials (e.g., at the filling margins, the area surrounding a dental implant, the area underneath a crown, etc.). The presence of arginine will support the growth of such arginolytic, which are healthy bacteria that produce ammonia via arginine metabolism such as, but not limited to, S. gordoni and S. sanguinis. Ammonia production can neutralize the glycolitic acids produced by caries pathogens and lead to an increased local pH. The increased pH in these hard to reach areas will help to suppress the growth of S. mutans and other caries pathogens that thrive in acidic environments.

Thus the present disclosure provides restorative dental materials containing arginine, which is released from the dental materials over time. This disclosure also provides methods of making the arginine-containing restorative dental materials as well as methods of using the arginine-containing restorative dental materials of the present disclosure to prevent, control, and/or reduce caries activity in a host, particularly secondary caries associated with/adjacent to a restorative dental material.

Embodiments of the present disclosure provide a restorative dental material for use in various restorative dental practices (fillings, sealants, varnishes, crowns, caps, bridges, dental bonding, endodontic materials, and the like). In embodiments, the restorative dental materials of the present disclosure include a restorative dental composition and arginine, combined such that the arginine is released into the oral cavity of the host over time. In addition to arginine, the restorative dental composition can include any composition or the ingredients of a known composition of a typical restorative dental material (such as that used to provide restorations (e.g., fillings)) placed in teeth present in the oral cavity of a host. The ingredients and arginine are combined such that at least a portion of the arginine is released over time from the composition into the surrounding environment (e.g., the oral cavity of the host). Typically, in restorative dental practice, restorative dental materials are intended to be permanent or semi-permanent. In other words, in contrast to temporary oral hygiene products like pastes, gels, and rinses used in dental hygiene or other temporarily applied products, the restorative dental materials of the present disclosure are applied/installed into the oral cavity of a host (e.g., on or adjacent to tooth surfaces) with the intent that they remain in the hosts' mouth over a longer period of time, such as days, weeks, years, many times for the remaining life of the patient. Thus, in embodiments, the arginine is incorporated in the restorative dental material such that it is released over a period of time. For example, as described in greater detail Example 1, difference concentrations of arginine were incorporated into a dental adhesive/bonding material by mixing the arginine with known polymerizable monomers used in current commercially available dental adhesives.

In embodiments, the arginine is incorporated in a manner and amount that does not interfere with the desired mechanical and physical properties of the restorative dental material, such as strength, flexibility, hardness, and the like. The arginine can be incorporated into various commercially available restorative dental materials, the restorative dental compositions, and/or ingredients used to form restorative dental materials (e.g., prior to a hardening, curing, or polymerization steps). For instance, the arginine can be mixed into a pre-mix, kit, or combination of components/ingredients for a restorative dental composition (either one that is commercially available, or made from raw materials) and then further processed to form a restorative dental composition/material. Further processing may include combining with an activator, polymerizing (e.g., chemically or via heat, UV light, etc.), molding, etc. The ingredients used to form the restorative dental materials can include raw ingredients obtained separately or commercially available combinations, pre-mixes, or kits of ingredients for specific restorative dental materials. Various concentrations of arginine can be incorporated depending on the properties of the material and the desired amount of arginine. In embodiments, the arginine is about 2 to 15 wt % of the restorative dental material. In yet other embodiments, the arginine is about 5-10% by weight of the restorative dental material, or about 5-7% by weight of the restorative dental material. Embodiments of the present disclosure contemplate other ranges as well as overlapping and intermediate ranges in addition to those listed above.

The arginine is released from the restorative dental material into the oral cavity of a host. The range of arginine release in vivo can vary depending of many factors such as degree of saturation of saliva and dental plaque, pH of saliva and plaque, and others. In some embodiments, the arginine is released at a rate of about 0.2 μmol/cm² to about 75.0 μmol/cm² μmol/cm². In some embodiments the initial release is high, such as 70.0-75.0 μmol/cm² (see Example, below), and then diminishes somewhat after about 24 hrs to about 3.0-5.0 μmol/cm², and after about 48 hrs. to about 3 d to about 1.0-3.0 μmol/cm². In embodiments, the release leveles off after an initial period (e.g., about 3 days) to a range of about 0.5 to 1.0 μmol/cm², such as about 0.7 μmol/cm². In embodiments, the release levels level off to a range of about 0.2 to about 1.0 after about 7 days.

In embodiments, the restorative dental material of the present disclosure includes, but is not limited to, dental adhesives, dental bonding materials, resin cements, resin composites, sealants, varnishes, resin modified glass ionomers, prosthodontic and orthodontic appliances, customized trays for dental whitening, and mouthpieces for caries/erosion and/or bite alignment purposes (e.g., night guards, bite guards, etc.). In some embodiments, the arginine-containing restorative dental material includes a dental adhesive/composite/bonding material (e.g., that used in fillings, sealants, dental bonding, and the like). An example of such dental adhesive material is described in the Examples below. In embodiments, the dental adhesive material includes monomeric materials that are capable of polymerizing (e.g., by application of UV light, heat, initiator or accelerator compound, etc.) to form the dental adhesive material, and the arginine can be mixed with the monomeric material and any optional filler materials prior to polymerization and will be incorporated into the formed dental adhesive material upon polymerization. In embodiments, the dental adhesive material includes monomers including, but not limited to, methacrylate monomers, acrylate monomers, and combinations of these, where the monomers polymerize to form an adhesive bond. In embodiments, the dental adhesive material can also include a variety of oligomers and monomers, such as but not limited to vinylphosphonate, that can solidify to form strong adhesive bonds.

In embodiments, the restorative dental material also includes a filler material. In embodiments, the filler material can include, but is not limited to, silanized glass, amorphous and/or colloidal silica, polyacrylic acid polymers, ceramics, quartz, organically modified ceramic, and combinations of these materials. In embodiments, the material is a dental adhesive and the filler comprises silanized glass.

In embodiments, the restorative dental material is a dental adhesive composition for use in restorative dentistry and includes a polymerizable compound or mixture of compounds capable of polymerizing to form an adhesive bond, an optional filler material, and arginine, where the arginine is incorporated into the dental adhesive composition (or formed dental material) and is released from the dental adhesive composition/formed dental material into an oral cavity of a host over time. In embodiments, the polymerizable compound is selected from methacrylate monomers, acrylate monomers, or combinations thereof. In some embodiments, the filler is silanized glass. In some embodiments of a dental adhesive according to the present disclosure, the arginine makes up about 2-10% by weight of the dental adhesive composition.

In embodiments, the arginine in the dental materials of the present disclosure can be in a variety of forms, including, but not limited to: free arginine (L-Arg), arginine peptides, polyarginine, arginine salts, hydrolysable esters of arginine, and the like, and combinations of the above.

Although the examples below describe several formulations of a dental adhesive composition modified to include various concentrations of arginine, other restorative dental materials can be similarly re-formulated to add arginine according to the descriptions and methods of the present disclosure.

In addition to the restorative dental materials described in this disclosure, methods of reducing caries associated with restorative dental materials (such as, but not limited to secondary caries at restorative dental composite margins) in a host are also provided. In embodiments, such methods include using a restorative dental material including arginine in the oral cavity of a host, where the arginine is released from the restorative dental material into the oral cavity of the host over time. As discussed above, this sustained release of arginine helps to fuel the ADS activity of arginolytic bacterial cultures present in oral biofilms, thereby increasing ammonia production via the ADS, neutralizing the acid produced by caries pathogens, and therefore, raising the pH levels of the localized biofilms, which can inhibit and reduce the proliferation and activity of caries pathogens such as but not limited to S. mutans. The presence of the additional arginine in the oral environment of the host and the resulting increased ammonia production via arginolytic bacteria and consequent increase in pH can result in a shift in the ratio of arginolytic bacteria to cariogenic bacteria in the oral microbiome of the host.

In embodiments of the methods of reducing caries in a host, the caries associated with restorative dental materials is located around the margins of a dental filling, such as at the tooth composite interface. In embodiments, the arginine-containing restorative dental materials of the present disclosure are used to create the filling. Thus, the arginine in the restorative dental material of the filling increases the pH of oral biofilms located adjacent to the restorative dental material thereby inhibiting/reducing/preventing growth of cariogenic pathogens, such as S. mutans.

The present disclosure also provides methods of making restorative dental materials of the present disclosure that have anti-caries activity. In embodiments, the methods of making the arginine-containing restorative dental materials includes providing a restorative dental composition or the ingredients for a restorative dental material (e.g., polymerizable monomers or other ingredients, fillers, etc.) and combining arginine with the restorative dental composition or the ingredients to form the restorative dental material. The resultant restorative dental material has arginine incorporated into the restorative dental material, such that the restorative dental material releases arginine into an oral cavity of a host over time. This sustained release helps fight caries in the area surrounding and/or adjacent to the restorative dental material, including difficult to access areas.

In embodiments, the arginine in the restorative dental materials of the present disclosure may have the potential of being “recharged” with arginine if the arginine incorporated at the time of formation/implantation of the restorative dental material has been depleted or reduced to a point where the release rate is not as effective at counteracting cariogenic bacteria. In embodiments, the restorative dental material can be recharged with arginine by exposing the restorative dental material (whether located in the oral cavity of the host as in the case of a permanent restorative dental material (e.g., filings, crowns, etc.) or temporarily removed for the recharge procedure (e.g., intra-oral removable dental appliances, such as trays, night/bite guards, orthodontic appliances, dentures, etc.). In embodiments, an arginine containing composition (such as an oral rinse, paste, gel, micro- or nanoparticles, etc.) can be applied to/contacted with the restorative dental material in a sufficient amount and for a sufficient amount of time such that at least a percentage of the arginine in the composition is taken up by the restorative dental material, such that later it can be released into the surrounding oral environment over time. For instance, for non-removable dental materials, such as fillings, sealants, crowns, bridges, some orthodontic appliances, etc., recharge could be accomplished by using an arginine-containing rinse, paste, or gel that is placed in the oral cavity of the host in contact with the dental material for a period of time (e.g., by rinsing with the arginine composition, pasting or painting it on the material, using trays containing the arginine composition, etc.). In other embodiments, such as with removable dental materials (e.g., trays, dentures, guards, some orthodontic appliances, etc., the material can be removed and placed in or in contact with a composition (e.g., liquid, gel, micro- or nanoparticles, etc.) containing arginine for a period of time.

Additional details regarding the tests and methods of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following embodiments.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

The following examples describe the formulation of a new dental bonding agent/composite/adhesive containing arginine. This new dental adhesive will help to create a neutral environmental pH less favorable to the growth of caries pathogens, thus reducing the risk for caries at the tooth-composite interface. This example describes development and evaluation of an etch-and-rinse adhesive system containing four different concentrations of arginine for sustainable release and recharge without affecting mechanical properties of the adhesive system.

The arginine-based bonding agent was formulated to include the components of known dental composite compositions, e.g., methacrylate monomers and silanized glass fillers, with the addition of various percentages of arginine. The arginine-based bonding agent was fabricated with methacrylate monomers and silanized glass fillers, and tested for: (i) the mechanical properties of true stress, modulus of elasticity and bond strength, (ii) arginine release and recharge, and (iii) anti-caries activities.

Initial results demonstrate that arginine was released from the composite at a rate and concentration to exhibit anti-caries effects and that the inclusion of arginine, particularly at 7% did not adversely affect the adhesive/bonding properties of the composite. It is believed that the application of arginine-based bonding agents of the present disclosure will dramatically reduce the incidence and severity of secondary caries in composite restorations in an economical fashion.

### Example 1: Arginine-Containing Restorative Dental Adhesive Formulations

### Materials & Methods

Arginine-Containing Adhesive Formulation

Arginine was incorporated into known monomers to test various formulations of a dental adhesive system including a primer and a bonding agent. Arginine (C₆H₁₄N₄O₂) has a molar weight of 174 g/mol (1 gram C₆H₁₄N₄O₂ is equal to 0.00574 mole). To formulate a primer and an adhesive, the molarity itself or the total volume of the whole blend can be used. The latter was used in the present example.

The first arginine-containing dental adhesive system formulation was conducted using the following volume proportion: 40% HEMA ((hydroxyl ethyl methacrylate), 20% water, 40% HDDMA (1,6-hexanediol dimethacrylate monomer), and arginine in one of the following amounts: 0% (control), 2%, 5%, and 10%.

As arginine is less soluble in solvents such as water, one goal for this first formulation was to assess solubility and select a solvent to result in a well blended final solution. The following sequence was followed:

1. Add water;

2. Add arginine;

3. If arginine stays in solution, add HEMA;

4. Add HDDMA.

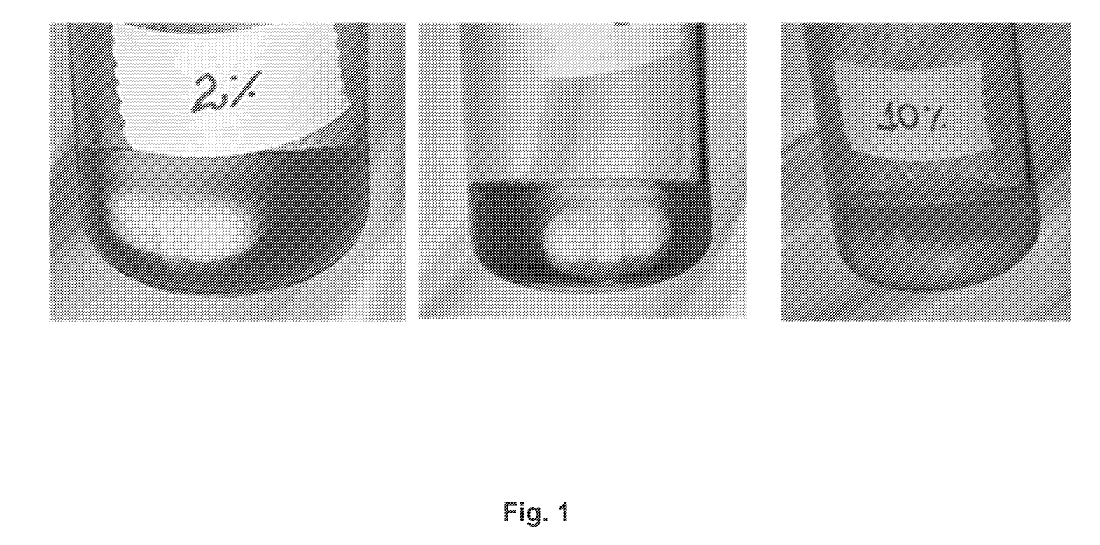

It was observed that when HDDMA was added last, a phase separation occurred as shown in FIG. 1 below. Based on this finding, new formulations were designed to avoid using HDDMA and still have an arginine acceptable dissolution.

A second formulation was designed without HDDMA while still allowing arginine dissolution. The second formulation was based on a three-step etch & rinse dental adhesive system.

The primer solution included (by weight percent):

ethoxylated bisphenol-A dimethacrylate (BisEMA) (15%)
hydroxyethyl methacrylate (HEMA) (10%)
urethane dimethacrylate (UDMA) (10%)
triethylene glycol dimethacrylate (TEGDMA) (10-15%)
Distilled water (15%)

### Ethanol (40%)

The solution was in the ratio of 45 wt % monomers and 55 wt % solvents.

The adhesive formulation included (by weight percent):

### UDMA (40%)

glycidyl methacrylate (BisGMA) (10%)

### Bis-EMA (17%)

### TEGDMA (30%)

camphorquinone (CQ) (0.5%)/amine (EDAB) (1%)
Diphenyliodonium salt (diphenyliodonium hexafluorophosphate (DPIHP)) (1.5%)
The monomer mixture previously homogenized in the ratio of 97 wt % of monomers and 3 wt % photo-initiator agents.

The arginine (L-arginine; Sigma-Aldrich) was incorporated by weight in the following different concentrations in the adhesive mixture:

0% arginine (Arg0; control)
2.5% arginine (Arg2.5)
5% arginine (Arg5)
7% arginine (Arg7)
10% arginine (Arg10)

L-arginine was added to the adhesive mixture and homogenized. The adhesive system was prepared in a dark room under controlled temperature and humidity and kept under refrigeration at 4° C. until use. Prior to use, the above mixed adhesive system was stirred for about 15 minutes.

Three-Point Bending Flexural Test—Flexural Strength (FS) and Flexural Modulus (FM)

Flexural strength and elastic modulus tests were conducted on bar-shaped specimens (n=7 per study group) created with 5 different concentrations of the three-step etch & rinse adhesive system containing arginine (control (0% arginine), 2.5%, 5%, 7%, 10%). Tests included a three-point bending flexural test. The flexural test was performed according to ISO 4049 (incorporated herein by reference), except for the specimen's dimensions (1 mm length×2 mm width×2 mm thickness). Each experimental adhesive was inserted into pre-manufactured silicon impressions (molds). A polyester strip was seated on surface of the specimen and pressed manually to remove excess adhesive. A glass cover slip was also placed on top of the polyester strip to avoid incorporation of air bubbles in the specimen. The adhesive was light-cured with a LED light-curing unit (Valo, Ultradent, USA) at 1,000 mW/cm² for 20 s. After photo-activation, the specimens were stored in an incubator at 37° C.±1 for 24 h±1, in a dark and dry container.

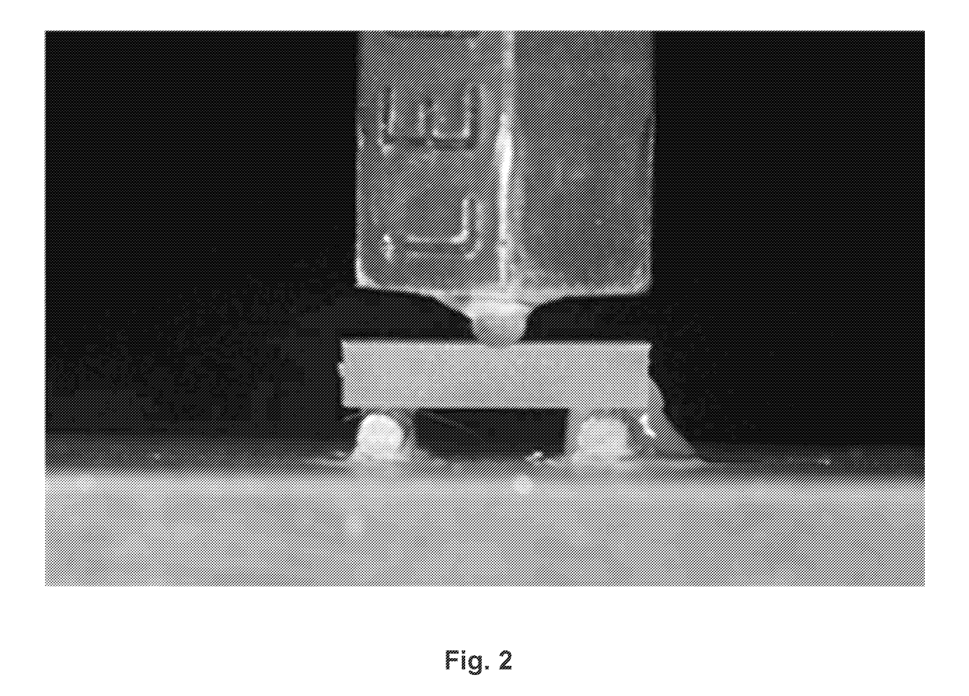

The three-point bending test was performed in a universal testing machine (Instron, Canton, USA—span between supports=5 mm) at a cross-head speed of 0.5 mm/min (FIG. 2). The maximum load for the specimens at fracture was recorded, and the flexural strength (FS) was calculated using the following equation: FS=3FL/(2bh²), where F is the maximum load (N) exerted on the specimens; L is the distance (mm) between the supports; B is the width (mm) of the specimens measured immediately prior testing; and H is the height (mm) of the specimens measured immediately prior testing. FM data was obtained according to the first load-displacement curve of the linear portion from the graphic provided by the BlueHill 3 software built into the Instron testing machine.

The flexure strength of each specimen, N/mm2 (σ), was calculated from the equation: σ=3FL/2bh², and the elastic modulus, N/mm2 (E) was determined from the equation: E=L3 m/4bh³d, where F is the load (N) at a given point in Newton (N), L is the distance between the support span (mm), b is the breadth of specimen (mm), h is the height of specimen (mm), and d is the maximum flexure.

The elastic modulus was measured as the slope of the strain×strain curve in the linear portion, using the following equation: E=L1D310−3/4BH³D, where L1 is load (N); 28D: distance (mm) between the supports; B: width (mm); H: height (mm); D: displacement (mm). Data were analyzed by two-way ANOVA and post hoc Tukey's test. Statistical significance was established at α=0.05.

Knoop Hardness (KHN)

Circular specimens (n=5 per study group) were prepared by placing each experimental adhesive into a rubber mold of 5×1 mm (Odeme Dental Research, Luzerna, SC, Brazil) and light-curing at 1,000 mW/cm2 for 20 s (Valo, Ultradent, USA). Specimens were kept stored for 24 h in dry conditions at 37° C. in a dark and dry container and embedded in polystyrene resin for grounding and polishing. Next, the top surfaces were ground and polished under water using 320, 400, 600 and 1200 grit sandpapers (Carborundum) on an automated polishing machine under water-cooling at all time to obtain a polished surface. The specimens were than dried and submitted to Knoop hardness measurements in a microhardness tester HMV-2 (Shimadzu, Tokyo, Japan) with a load of 50 g and dwell-time of 15 s in order to obtain five measurements from each specimen. T For each specimen, five readings were taken, and an average of them is calculated. The data were submitted to two-way ANOVA, followed by Tukey's test at the 5% significance level (see Table 1). The mean KHN value was obtained by averaging the five indentations.

Cohesive Strength Test/Ultimate Tensile Strength (UTS)

Specimens (n=10 per study group) were prepared using silicon molds with an hourglass shape of 10×4 mm and a cross-sectional area of 1.5 mm² (Odeme Dental Research, Luzerna, SC, Brazil). The molds were used to make each hourglass samples of each experimental adhesive arginine concentration except the 2.5% arginine. Each arginine-containing adhesive was inserted in the silicon mold, and a transparent strip (Myler strip) placed on top of each matrix, and light cured using a LED light-curing unit at 1,000 mW/cm² for 20 s (Valo, Ultradent, USA). These hourglass samples were stored for 24 h in 37° C. Specimens were fitted in a testing jig device and submitted to tensile strength test. Load was applied perpendicular to the plane of the cured adhesive in a semi-universal testing machine OM100 (Odeme Dental Research, Luzerna, SC, Brazil) and tested as ultimate tensile strength at a speed of 0.75 mm/min The cohesive strength, in MPa, was calculated using the following formula: CS=F/A, where F is tensile strength (N), A is was the sample transversal cross section area (mm²).

Degree of Conversion (DC)

Circular specimens (n=5 per study group) were prepared in the same manner as for Knoop hardness test and evaluated immediately after light-activation. DC was determined by a Fourier Transform Infrared spectrometer (Tensor 27, Bruker Optics GmbH, Ettlingen, Germany), coupled to an attenuated total reflectance (ATR). Absorbance spectra included 16 scans at a resolution of 1 cm−1. Unpolymerized blends were scanned after been placed into a Teflon mold (ϕ=5 mm, 1 mm thick) and taken to the ATR. The adhesive blends were light-cured through a polyester strip using a light-curing unit (Valo, Ultradent, USA) for 20 s at 1,000 m/Wcm2. The polymerized samples were then scanned, and unconverted carbon double bonds were quantified by calculating the ratio derived from the aliphatic C═C (vinyl) absorption (1638 cm−1) to the aromatic C═C absorption (1608 cm−1) peaks for both polymerized and unpolymerized samples. The DC for each resin was calculated according to the follow equation: DC (%)={1−(Xa/Ya)/(Xb/Yb)}×100, where, Xa (polymerized) and Xb (unpolymerized) represent the bands of the polymerizable aliphatic double bonds, and Ya (polymerized) and Yb (unpolymerized) represent the bands of the aromatic double bonds.

Statistical Analysis

All data management was performed using SAS procedures (SAS 9.1.3). For descriptive analysis, distribution of percentages and means were calculated when appropriate. T-test or ANOVA were used to test the differences of continuous variables; and chi-square test was used for categorical variables, with a significance level of 95% (α=0.05).

### Results

Flexural Test & Elastic Modulus Test

Results of the three point bending flexural test (n=7) elastic modulus test (n=7) of 5 different concentrations of an three-step etch & rinse dental adhesive system containing arginine (control, 2.5%, 5%, 7%, 10% arginine) are presented in the tables below.

TABLE 1
Flexural strength, flexural modulus, Knoop hardness and cohesive
strength/ultimate tensile strength of experimental arginine-containing
adhesive system tested in different concentrations.

| | | Flexural | | | |
|---|---|---|---|---|---|
| Adhesive | Ultimate Tensile | strength | Flexural | Knoop | Degree of |
| System | Strength (MPa) | (MPa) | modulus (GPa) | hardness (KHN) | conversion (%) |
| Control | 28.87 (±7.75)^{a} | 115.36 | 1.24 (0.17)^{a} | 23.40(1.22)^{a} | 54.82 (0.82)^{a} |
| | | (21.43)^{a} | | | |
| 2.5% Arginine | 28.21 (8.34)^{ab} | 91.05 | 1.18 (0.04)^{a} | 23.25 (1.08)^{a} | |
| | | (6.33)^{b} | | | |
| 5% Arginine | 25.22 (6.81)^{bc} | 91.81 | 1.13 (0.22)^{a} | 21.88(1.66)^{a} | 55.34 (2.58)^{a} |
| | | (11.83)^{b} | | | |
| 7% Arginine | 27.03 (723)^{ab} | 95.74 | 1.26 (0.12)^{a} | 23.05(0.94)^{a} | 55.55 (1.22)^{a} |
| | | (10.61)^{ab} | | | |
| 10% | 21.72 (5.95)^{c} | 89.58 | 1.29 (0.23)^{a} | 20.38(0.85)^{a} | 57.12 (2.60)^{a} |
| Arginine | | (10.41)^{b} | | | |

Means followed by same small letter in the same column are not statistically different at 5%, by Tukey's test.

For FS, Ag0 had the best mean when compared to Arg5 and Arg10 groups (p<0.05). Arg7 group showed intermediate mean and did not differ from other groups (p>0.05). The FM results illustrate that different concentrations of arginine incorporated to the tested dental adhesive system did not have statistical significant difference among all groups, including Control, when elastic modulus what evaluated (p>0.05). There were no statistically significant differences for Knoop hardness evaluation, therefore indicating that the addition of arginine, regardless of the concentration, does not appear to affect polymerization of the proposed formulation.

For UTS, the control group (Arg0) had the best mean when compared to Arg5 and Arg10 groups (p<0.05). Arg7 group did not differ from the other groups (p>0.05), except for Arg10. For FS, Ag0 had the best mean when compared to Arg5 and Arg10 groups (p<0.05). Arg7 group showed intermediate mean and did not differ from other groups (p>0.05). There was not statistical difference among the groups for E and for KHN (p>0.05).

### Conclusions

The incorporation of arginine at all concentrations tested did not alter the flexural modulus, and the incorporation of 7% arginine did not alter the flexural strength of the experimental adhesive as compared to the control.

### Example 2: Microtensile Bond Strength Test (UTBS)

After testing all concentrations of arginine adhesives (Control, 2.5%, 5%, 7% and 10%) under thee point bending flexural strength test and observing elastic modulus analysis as set forth in Example 1, above, the concentrations of Control (0%), 5% 7% and 10% were chosen for dentin micro-tensile bond strength (μTBS), so each one could be tested in a different dentin quarter as described below.

### Materials and Methods

Sample Characterization and Preparation

Under approval of the University's Institutional Review Board (IRB: 201500052), thirty (30) intact non-carious and non-restored human third molars were collected from the Department of Oral Surgery, College of Dentistry, University of Florida. Teeth were stored in 0.5% chloramine-T solution at 4° C. and used within 3 months following extraction without any possibility of individual or gender distinction among them. No researchers involved had any kind of direct contact with the patients or the surgery procedure.

Teeth were cleaned from soft tissues and calculus with periodontal curette 5-6 (Duflex, SS White, Rio de Janeiro, RJ, Brazil), followed by prophylaxis with slurry containing water and pumice. After cleaning, teeth were stored in water until the sectioning/bonding procedures.

Criterion for tooth inclusion in this research was the following: sound third molars with complete root formation. The exclusion criteria were the following: third molars with any type of malformation and/or carious lesion, and/or sound third molars with incomplete root formation.

Bond Strength Analysis

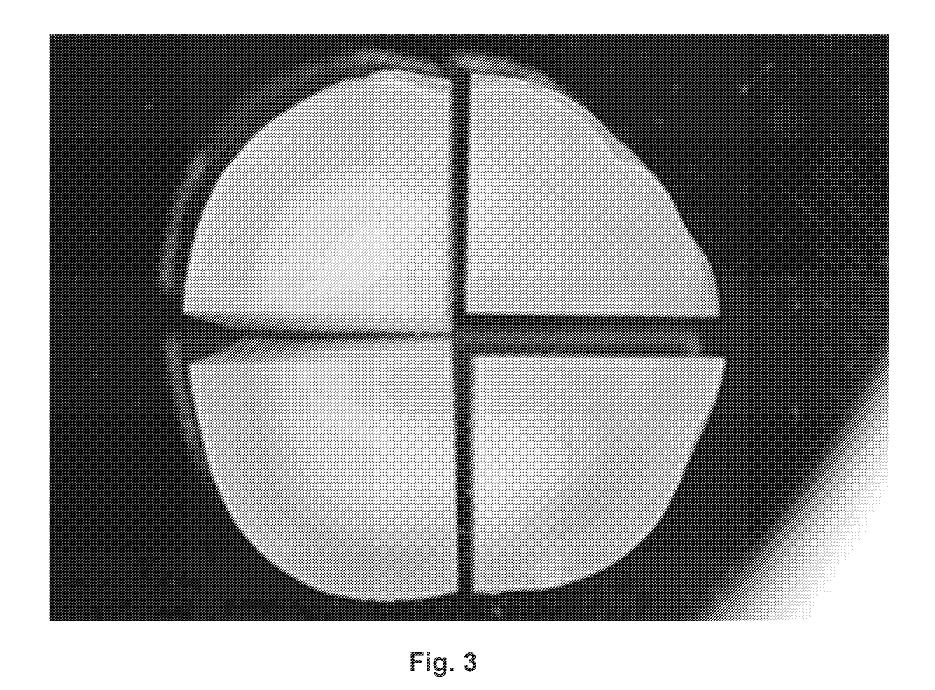

A primary section was performed perpendicular to the long axis of the tooth to remove the occlusal enamel and expose a large area of middle dentin surface. Then, a second section 3 mm below the cement-enamel junction was performed to remove roots. The sections were performed using a cutting machine (Isomet 1000, Buehler, Lake Bluff, Ill., United States) with diamond double side disc at a low speed (150 rpm) and under continuous water-cooling. Thereafter, each crown was sectioned into four parts (Geraldeli et al., 2002) (FIG. 3). Each quarter was randomly assigned to the following groups (n=30 per group): Arg0, Arg5, Arg7 and Arg10. Thus each group was tested on a section of the same tooth. In total there were 30 fragments (quarters) for each arginine adhesive concentration (n=30). While a quarter was being treated with the respective adhesive, all other quarters were protected by a thick Teflon tape.

Before applying the arginine-contained dental adhesive (control, 5%, 7% and 10%) to the dentin structure according to a pre-standardized technique, the exposed dentin surface was slightly grinded by hand doing an “8” shape on a sandpaper (600-granulation silicon carbide) under water for 30 s to simulate the smear layer formation caused by a diamond bur preparation. This substrate was acid etched with 37% phosphoric acid for 15 s, rinsed for the same time and air-dried leaving the dentin moist. A primer solution was actively applied using microbrush for 10 s followed by a very gentle air-dry (15 cm from the substrate for 5 s) aiming to evaporate the solvent (solvent concentration in tables from Example 1, above).

The adhesive was actively applied during 10 s and gently air-dried. Light curing was carried out during 10 s using a LED light-curing unit (Valo, Ultradent, USA). After that, using a micro-hybrid resin based composite Filtek Z250 (3M ESPE, St. Paul, United States), a first layer of 0.5 mm was carefully placed and light cured for 20 s, avoiding any kind of contact between the plastic instrument used and the adhesive layer just light cured. Three additional composite layers of 1.5 mm were placed using the incremental technique and curing each layer between applications. This produced four incremental layers, the first one having a thickness of about 0.5 mm and the other 3 having a thickness of about 1.0 mm each. Each layer was light cured for 20 s with the light source LED Bluephase G2 (Ivoclar Vivadent-, Schaan, Liechtenstein).

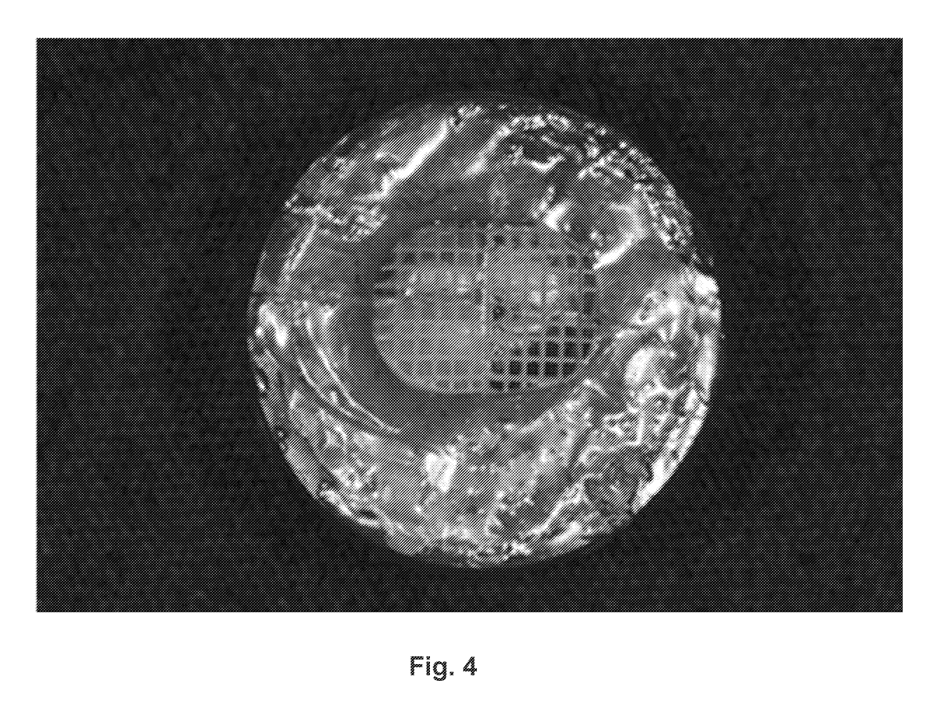

Subsequently, each restored dentin-quarter were painted according to the sharpie pen color used to identify them according to the arginine concentration used, as illustrated in FIG. 4. Each quarter was then cut into beams of ±0.9×0.9 mm² using the cutting machine (Isomet 1000, Buehler, Lake Bluff, Ill., United States) with diamond double side disc at low speed and constant cooling under water. Then, the beams obtained in each sample from each group were divided into four subgroups. Samples obtained were stored in labeled Eppendorf's tubes with water at 37° C. for 24 hours before microtensile bond strength test.

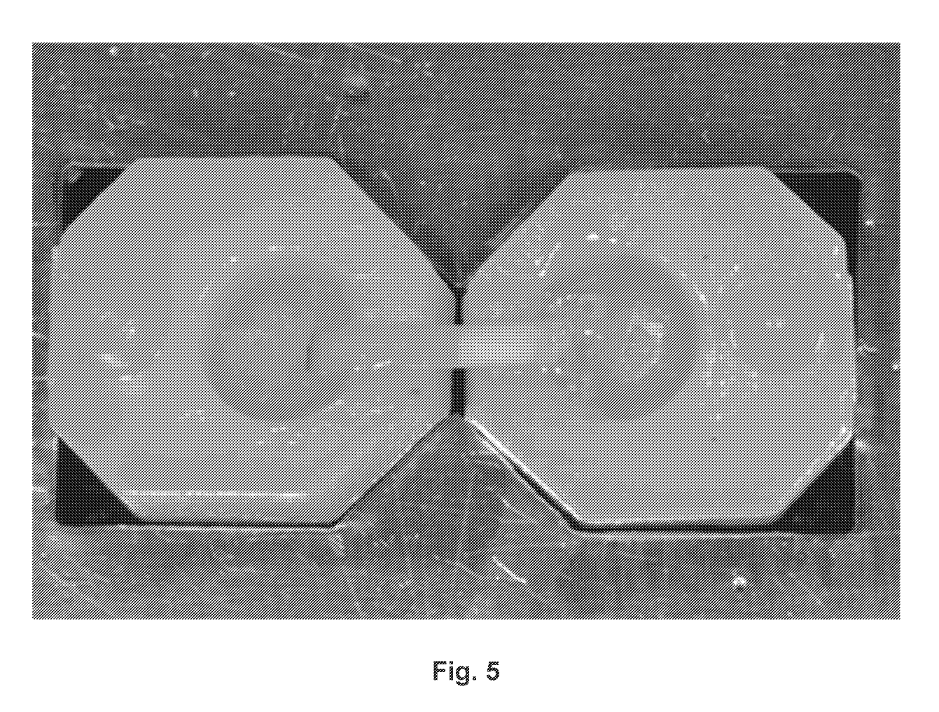

Before breakage during test, samples were carefully removed from Eppendorf's tubes, and the bonded interface area of each beam was individually measured with a digital caliper to an accuracy of 0.01 mm. To evaluate bond strength, beams of each group were taken to a portable microtensile testing machine OM100 (Odeme Dental Research, Lucerne, SC, Brazil), glued in the Geraldeli's V2 device (FIG. 5) and tested under tensile strength in a speed of 1 mm/min, using a 500N load cell until specimen rupture occurred. The stress required to cause specimen breakage was determined by the ratio between load (kgf) at the time of fracture and the cross section area of the specimen in mm². Beams from the same restored quarter had bond strength values grouped and the mean bond strength of each sample was regarded as a statistical unit. Beams that had early fracture (or fractured before the microtensile bond test itself) were excluded from the statistical analysis.

Final values was expressed in MPa considering the bonded area. The microtensile bond strength (μTBS) of each quarter was defined as the average of the tested beams. Failure mode was determined by evaluating each beam with stereomicroscope (50×, Nikon, model SMZ-1B, Japan) as described in Raposo, et al. 2012, Geraldeli and Carmo et al., 2002, and Geraldeli, S P and Larson, W K, 2002, which are incorporated by reference herein.

Fracture Pattern Analysis

After the microtensile bond test, the type of fracture can be further analyzed with a stereoscopic magnifying glass on an increase of 60×. Fracture patterns was further classified into the following categories: adhesive (A) when the fracture occurs only in the adhesive interface (hybrid layer and adhesive layer); cohesive within dentin (CD) when there is fully cohesive failure in dentin; cohesive within resin composite (CR) when fully cohesive failure occurs in restorative composite; and mixed pattern (M) when partial failure occurs at the bonded interfaces and in the cohesive composite or dentin.

Morphology of the Resin-Dentin Interface

Samples representatives from each study group were polished, conditioned and air-dried overnight. Samples were sputter-coated with gold-palladium for 60 s at 45 mA in a vacuum metalizing chamber (MED 010; Balzers, Liechtenstein) and the resin-dentin interfaces were examined in a scanning electron microscopy (LEO 435 VP; Carl Zeiss, Jena, Germany), operated under 20 kV.

### Results

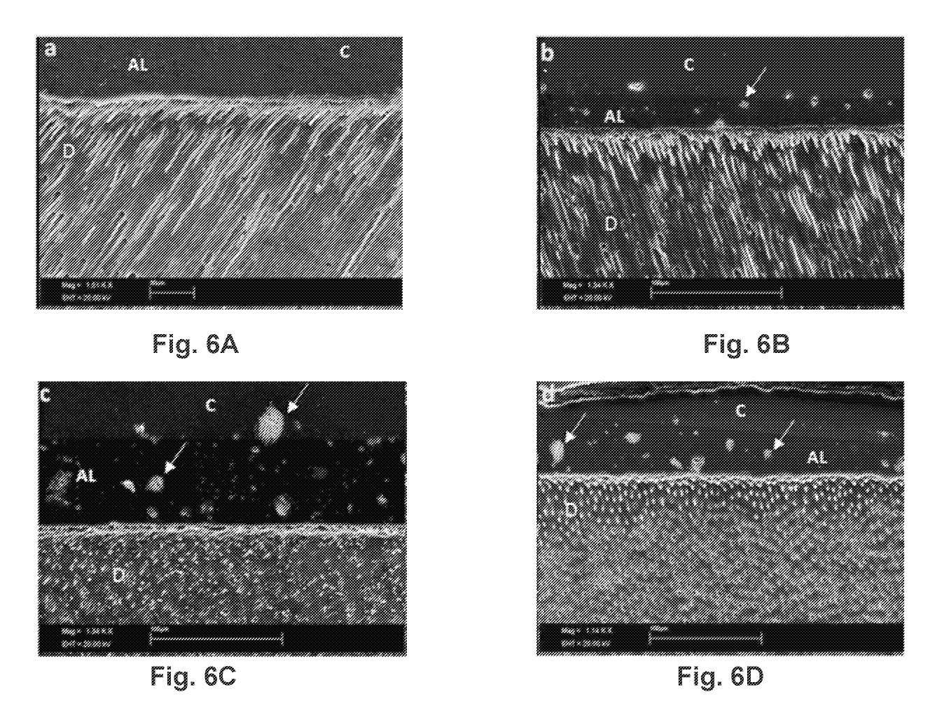

The μ-TBS means and standards deviations (MPa), as well as the failure pattern for all experimental groups are presented in Table 2. FIG. 6 shows the SEM images illustrating morphological aspects of the resin-dentin interface created with the adhesive systems formulated on this study.

TABLE 2
μ-TBS means for arginine-containing dental adhesive in
different concentrations.

| | | Failure mode (%) | | | |
|---|---|---|---|---|---|
| | | | | Cohes. | Cohes. |
| Material | Mean (MPa) | Adhesive | Mix | Dent | RC |
| Control | 15.09 (±8.80)^{a} | 26.6 | 66.7 | 0.0 | 6.7 |
| 5% Arginine | 14.55 ± 8.48^{a} | 53.4 | 40.0 | 3.3 | 3.3 |
| 7% Arginine | 13.48 ± 7.72 ^{a} | 33.3 | 60.0 | 0.0 | 6.7 |
| 10% Arginine | 11.92 ± 7.18^{a} | 66.7 | 33.3 | 0 | 0.0 |

Means followed by same small letter are not statistically different at 5%, by Tukey's test.

One-way ANOVA and Tukey test showed that there was not statistical difference among all groups (p>0.05). Yet, the adhesive failure was most common for the Arg5 and Arg10 groups. For Arg0 and Arg7, the mixed failures were the most prevalent.

### Conclusion

Concentrations of arginine ranging from 5% to 10% when added to experimental adhesive formulation did not affect the dentin bond strength when compared to 0%.

### Example 3: Arginine Release

### Methods

Arginine Release and Recharge

For arginine release, disc specimens with a diameter of 10 mm and a thickness of 1.2 mm and containing either ArgO or Arg7 adhesives were light-cured on both sides for 20 s each. Specimens (n=3 per group) were immersed in 2 mL high-purity water at 37° C. as previously described (Zhang et al., 2014) with some modifications. A 2-mL quantity of equilibrated water was taken at 0, 2 h, 4 h, 8 h, and then every 24 h up to 10 days, and finally at 30 days. The concentration of Arg released in the solution was analyzed by Liquid Chromatography Mass Spectrometry (LC-MS/MS) using [¹⁸O₂] arginine as internal standard. Samples were chromatographed on a Synergi Fusion (50×4.6 mm) column (Phenomenex) eluted with a linear gradient at 600 μl/min. The mass spectrometer was a Thermo-Finnigan TSQ Quantum Ultra utilizing electron spray ionization (ESI) and tandem mass spectrometry (MS/MS) in positive mode. Arginine concentrations were determined by calculating the peak area ratios of the arginine released to the standard, and comparing it to standard curves prepared using known concentrations of the authentic standards. For arginine recharge, the disc specimens were further immersed in large amounts of water and sonicated to reduce the amount of arginine. The specimens were then immersed in 2 mL of 1.5% L-arginine (same concentration as marketed arginine-toothpastes) aqueous solution for 1 min, rinsed for 10 s, carefully dried, and then placed in 2 mL high-purity water at 37° C. (replaced every 12 hrs); this procedure was repeated twice a day (every 12 hrs) and continued for 3 days. The collected solution was also analyzed by LC-MS/MS as described above.

Antibacterial Activity

The effects of the adhesives Arg0 and Arg7 on bacterial growth and biofilm formation were evaluated as previously described (Zhang et al., 2014) with some modifications. Planktonic growth Streptococcus mutans U159 and Streptococcus gordonii DL1 was tested with Bioscreen CTM (Oy Growth Curves AB Ltd, Helsinki, Finland). Ring specimens (inner diameter 3.1 mm, outer diameter 5.2 mm, thickness 1.1 mm, n=3) were made by light-curing the adhesives for 20 sec each side in a Teflon ring mold. After ultraviolet (UV) sterilization, the specimens were laid at the bottoms of 100-well honeycomb microtiter plates of Bioscreen CTM. Overnight cultures of the tested strains in Brain Heart Infusion (BHI) broth were transferred to fresh media and grown until mid-exponential phase (OD₆₀₀=0.5) in a 5% CO₂ aerobic atmosphere at 37° C. The cultures were diluted 1:100 into fresh BHI broth at pH 7.0 or 5.7, and aliquots (250 μL) were applied to the honeycomb plates. Bacterial growth was monitored every half-hour continuously for 24 hr with Bioscreen CTM with moderate shaking for 10 sec prior to OD measurements (Bitoun et al., 2011).

Confocal microscopy was used for analysis of biofilms. As previously done for planktonic assays, mid-exponential phase cultures of UA159 and DL1 were diluted 1:100 into fresh BHI broth at pH 7.0 or 5.7, and aliquoted into 8-well iBidi glass slides (vials) containing the adhesive discs. Biofilms were allowed to form for 48 hrs in a 5% CO₂ aerobic atmosphere at 37° C. with media resfresh after 24 hrs. Growth media and planktonic cells were removed after 48 hours and biofilm was washed gently with sterile phosphate-buffered saline (PBS). For analysis by confocal laser scanning microscopy, biofilms were stained with a LIVE/DEAD BacLight bacterial viability kit (Thermo Fisher), and the images were acquired using a spinning disk confocal system connected to a Leica DM IRB inverted fluorescence microscope that was equipped with a Photometrics cascade-cooled EMCCD camera. Syto9 fluorescence was detected by excitation at 488 nm, and emission was collected using a 525-nm (25 nm, green) band-pass filter. Detection of propidium iodide (PI) fluorescence was performed using a 642-nm excitation laser and a 695-nm (53 nm, red) band-pass filter. All z-sections were collected at 1-mintervals using a 63/1.40 oil objective lens. Image acquisition and processing were performed using VoxCell (VisiTech International, Sunderland, United Kingdom).

### Statistical Analysis

All data management and statistical analyses was performed using SAS procedures (SAS 9.1.3). For descriptive analysis, distribution of percentages and means were calculated when appropriate. T-test or ANOVA were used to test the differences of continuous variables; and chi-square test was used for categorical variables, with a significance level of 95% (α=0.05).

### Results

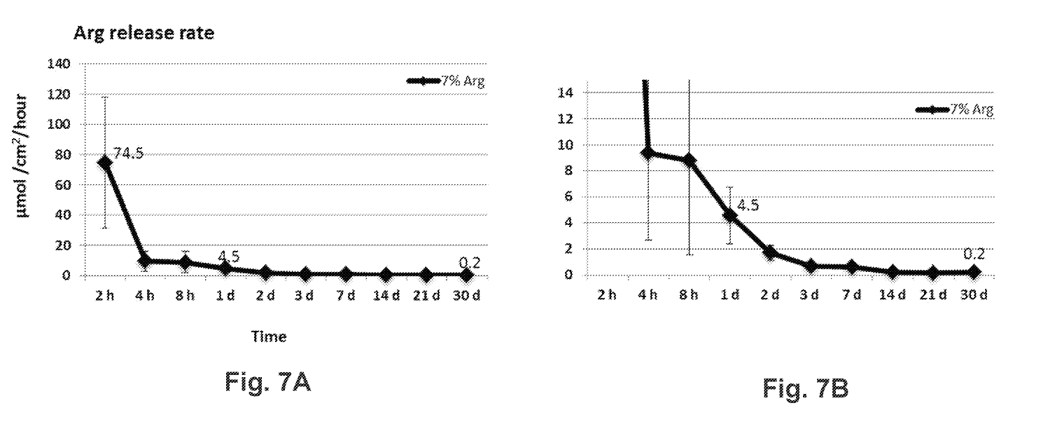

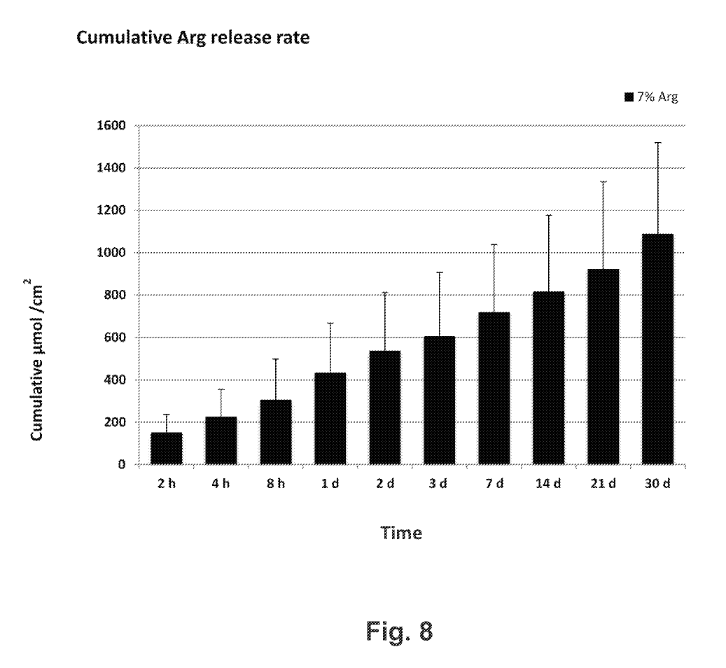

FIGS. 7A and 7B show the release rates of arginine from the tested adhesives over time (FIG. 7B is a detail of a section of FIG. 7A). FIG. 8 illustrates cumulative arginine release over time. The testing bonding agent containing 7% arginine showed a sustainable, controlled release of arginine for 30 days.

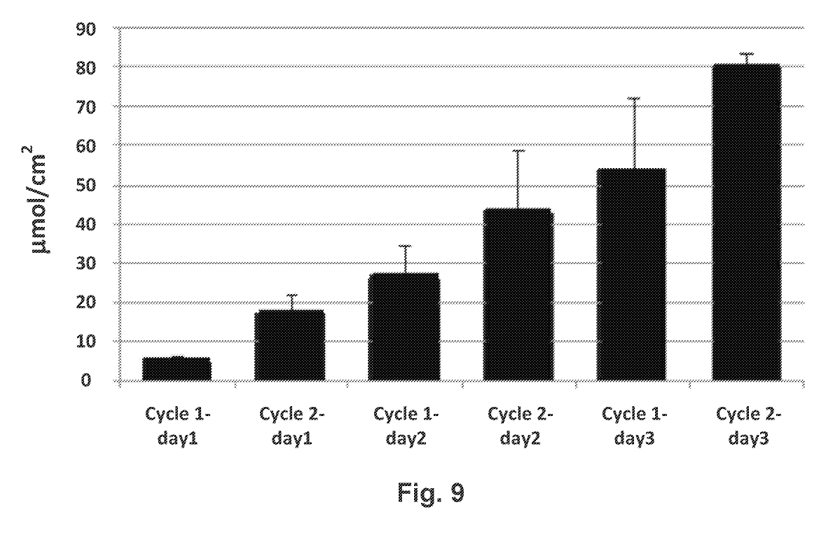

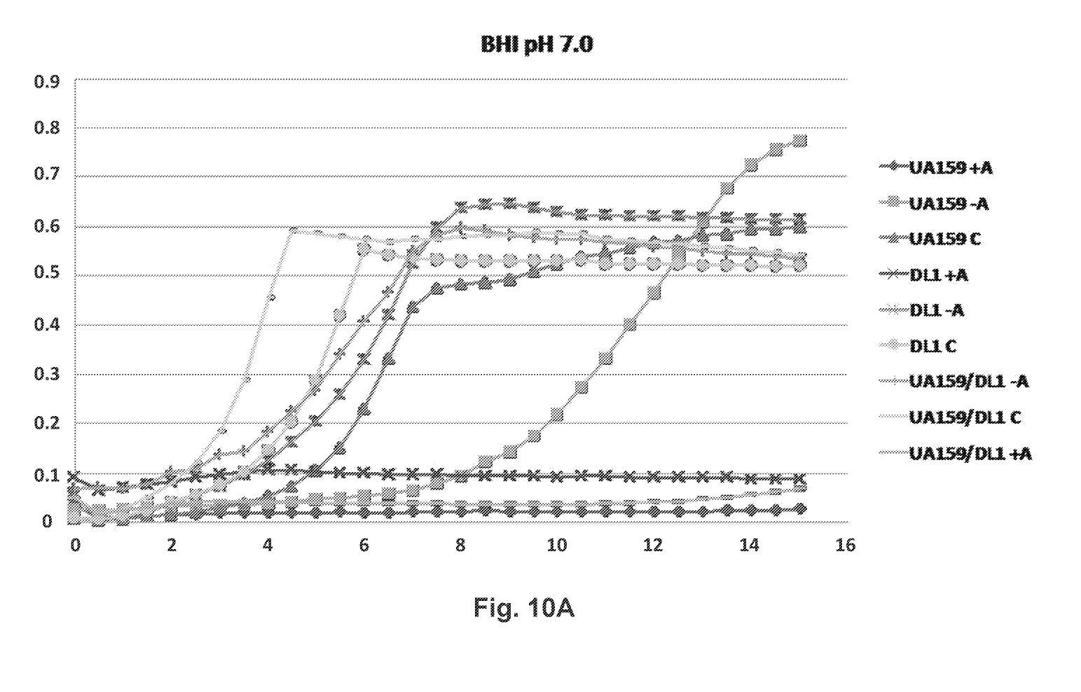

There was a high release rate in the initial 2 hours (74.5 μmol/cm²) after which the release quickly diminished after 24 hrs. (1 d=4.5 μmol/cm²) and 48 hrs.(2 d=1.7 μmol/cm²) and the release leveled off after three days (0.7 μmol/cm²) (FIGS. 7A & 7B). A total of 1086.7 μmol/cm² of arginine was released after 30 days (cumulative release, FIG. 8). This may be caused by dissolution and depletion of arginine particles on the polymerized adhesive system. However, it was shown in FIG. 9 that the adhesive system showed the capacity for being recharged with arginine.

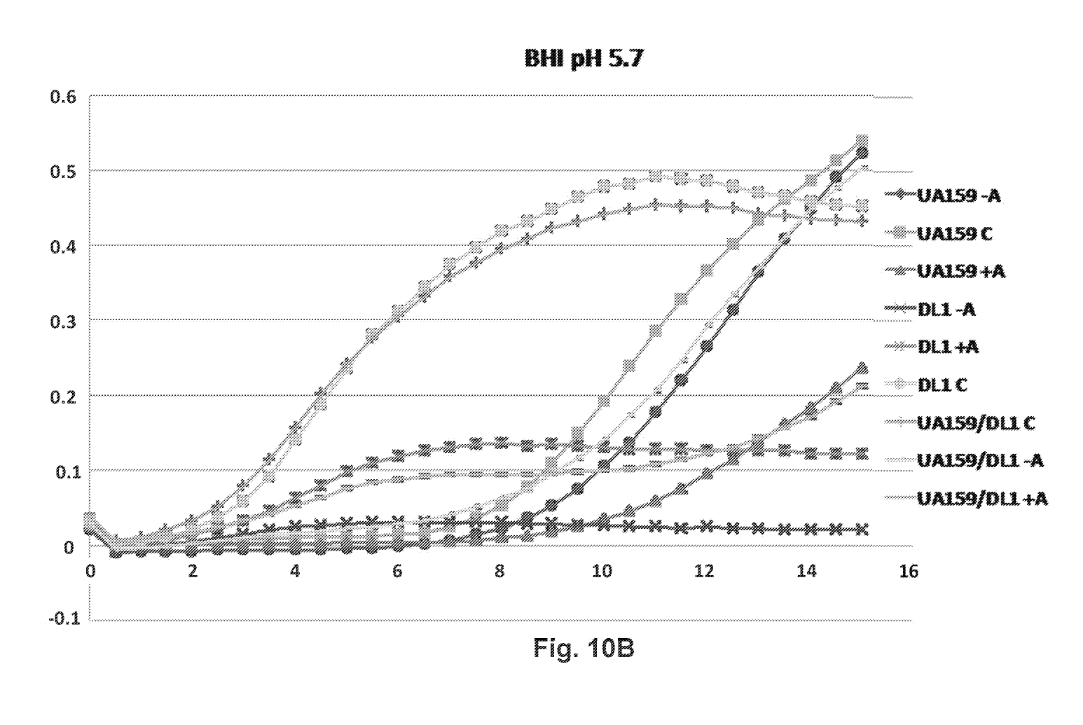

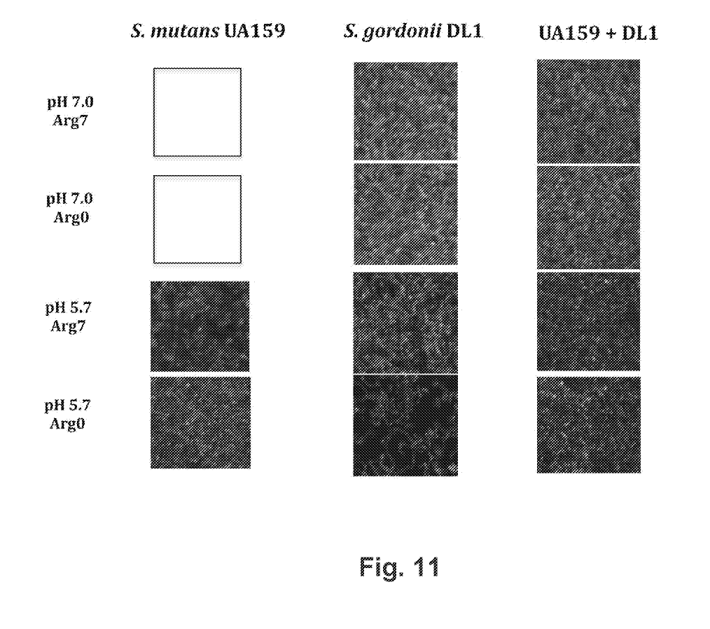

The planktonic bacterial growth of S. mutans UA159 and S. gordonii DL1 as single and dual cultures are shown in FIGS. 10A and 10B. At pH 7.0 (FIG. 10A), there was minimal or no bacterial growth in the presence of Arg7, and growth was also impaired in the presence of Arg0. Albeit slow, bacterial growth at pH 5.7 (FIG. 10B) appears not to be as affected by the presence of the tested adhesives. Low pH is known to induce ADS activity, and therefore aid in bacterial growth. Growth of cultures containing both UA159 and DL1 showed better resistance to the adhesive tested as compared to single cultures.

FIG. 11 illustrates the biofilm formed by UA159 and DL1 in BHI pH 7.0 and 5.7. UA159 in BHI pH 7.0 formed a very loose biofilm in the presence of Arg7 and Arg0, which was completely washed away in the preparation for the confocal microscopy, and thus no biofilm was observed under the microscope. In contrast, DL1 formed a sticker biofilm under the same conditions; the same was observed for dual species biofilms of UA150 and DL1. In general and consistent with the data of planktonic growth, less biofilm formation was observed in the presence of Arg0 and even less in the presence of Arg7 as compared to biofilm formation in the absence of the adhesives.

### Discussion and Conclusions

Despite significant technological improvements, current resin-based composite materials, including dental adhesives, are defenseless against bacterial bio-products. Clinically they require careful attention to the presence and/or risk of contamination. In addition, the vinyl polymerization of these materials leads to internal stresses, which can be transferred to the bonded interface creating discrepancies, cracks, and/or gaps. These materials do not have any buffering ability, which would reduce the amount of aciduric bacteria in the biofilm. Clinical trials have shown that new caries lesions develop at these margins, which accounts for the majority of replacement procedures. Thus, measures to reduce the occurrence of secondary caries at these margins are needed

In the dental field, arginine products have been applied as a desensitizing agent usually available in toothpastes (García-Godoy & García-Godoy, 2010; Boneta et al., 2013; Sharif et al., 2013; West et al., 2013, Yang et al., 2013 and Andreatti et al., 2014) and mouthwashes (Markowitz K, 2013 and Boneta et al., 2013), and they have been tested on enamel (García-Godoy & García-Godoy, 2010) and dentin surfaces (Canares et al., 2012, Wang et al., 2012 and Yang et al., 2013). However, incorporation of arginine in resin-based composite materials for caries reduction has not been suggested or proposed in the industry. This polar chemistry of arginine, based on carbon, nitrogen, oxygen, and hydrogen atoms and positively charged in physiological pH (pH 6.5-7.5) might be able to function as a caries activity inhibitor if incorporated into resin-based dental materials, if it does not otherwise interfere with the integrity and functions of the adhesive materials. Therefore, the above examples evaluated mechanical properties of an experimental adhesive system without (control group) and with different concentrations of arginine (5%, 7% and 10%).

The findings described above demonstrated that, for the first time, the addition of 2.5% to 7% arginine (and even 10% arginine) into a generic dental adhesive formulation did not compromise most of its mechanical properties. For UTS and FS tests, control and 7% arginine groups did not differ statistically. This finding indicates that an arginine-containing adhesive, at around range of arginine concentration, cannot only respond properly to the complex flexural stresses upon intra-oral loading of restorative materials, but also demonstrate anti-caries activity. Regardless of the concentration used, the adhesive had positive results in the adhesive for FM (flexural modulus) or stiffness, KHN (Knoop hardness) and DC (degree of conversion). It can be expected that the adhesive will perform well as to its light curing ability, stiffness and wear if used intra-orally.

For dentin microtensile bond strength test (μ-TBS), none of the concentrations exhibited a statistically significant difference (Table 2). Adhesive monomeric formulations such as the one used in the current adhesive systems have gone through a significant development process throughout the years aiming to achieve appropriate penetration into the demineralized dentin matrix (Landuyt et al, Biomaterials, Vol. 28, 26: 2757-3785, 2007, which is hereby incorporated by reference herein). The overall findings for dentin μ-TBS of this study were not quite comparable to commercial adhesives, but did demonstrate that the addition of arginine did not compromise the integrity of a similar adhesive.

The fact that arginine chemically interacts with calcium ions and that both components are present in dentin tissue did not affect μ-TBS. It appears that the incorporation of arginine into adhesives monomers did not prevent this amino acid from integrating homogenously into the adhesive layer and infiltrating into the dentin tubules. In addition, the mixed type of failure mode observed for 7% arginine supports the conclusion that hybrid adhesive layers were fully formed and behaved properly (Table 2).

Also of note is that in the scanning electronic microscopy images of the adhesive interface in FIG. 6, it can be seen that the acid-exposed collagen network was truly infiltrated by the adhesives from all groups. FIG. 6 shows that the arginine particles are fairly distributed in the adhesive layer, and more particles could be seen in the higher-arginine compositions. In addition, the smaller particles were able to penetrate within the adhesive and into dentin tubules in sporadic areas. Additional studies can be conducted to ensure that that arginine will be delivered from adhesive formulations at a rate and concentration to exhibit anti-caries effects regardless of ecological changes in the biofilm.

The results of the present examples support that the arginine-based adhesive system has the potential to: (i) retain the appropriate physical and mechanical properties, (ii) show controlled release and recharge of arginine over a prolonged period of time, and (iii) deliver arginine at a rate and concentration to exhibit anti-caries effects regardless of shifts in the biofilm environmental condition such as sugar availability and pH. Such approach has the potential to dramatically reduce the incidence and severity of secondary caries in composite restorations in a very economical fashion.

### REFERENCES

1. Ferracane J L. Resin-based composite performance: are there some things we can't predict? Dent Mater 2013; 29(1):51-8.
2. Gordan V V, Riley J L, 3rd, Geraldeli S, et al. Repair or replacement of defective restorations by dentists in The Dental Practice-Based Research Network. J Am Dent Assoc 2012; 143(6):593-601.
3. Imazato S, Ma S, Chen J H, Xu H H. Therapeutic polymers for dental adhesives: loading resins with bio-active components. Dent Mater 2014; 30(1):97-104.
4. Zhang K, Li F, Imazato S, et al. Dual antibacterial agents of nano-silver and 12-methacryloyloxydodecylpyridinium bromide in dental adhesive to inhibit caries. J Biomed Mater Res B Appl Biomater 2013; 101(6):929-38.
5. Nascimento M M, Burne R A. Caries prevention by arginine metabolism in oral biofilms: translating science into clinical success. Current Oral Health Reports 2014; 1(1):79-85.
6. Sharma S, Lavender S, Woo J, et al. Nanoscale characterization of effect of L-arginine on Streptococcus mutans biofilm adhesion by atomic force microscopy. Microbiology 2014; 160(Pt 7):1466-73.
7. Nascimento M M, Gordan V V, Garvan C W, Browngardt C M, Burne R A. Correlations of oral bacterial arginine and urea catabolism with caries experience. Oral Microbiol Immunol 2009; 24(2):89-95.
8. Nascimento M M, Liu Y, Kalra R, et al. Arginine metabolism may confer caries resistance in children. J Dent Res 2012; 91(A):691.
9. Zhang J F, Wu R, Fan Y, et al. Antibacterial dental composites with chlorhexidine and mesoporous silica. J Dent Res 2014.
10. Carrilho M R, Carvalho R M, Tay F R, Pashley D H. Effects of storage media on mechanical properties of adhesive systems. Am J Dent 2004; 17(2):104-8.
11. Carrilho M R, Tay F R, Pashley D H, Tjaderhane L, Carvalho R M. Mechanical stability of resin-dentin bond components. Dent Mater 2005; 21(3):232-41.
12. Raposo L H, Armstrong S R, Maia R R, et al. Effect of specimen gripping device, geometry and fixation method on microtensile bond strength, failure mode and stress distribution: laboratory and finite element analyses. Dent Mater 2012; 28(5):e50-62.
13. Geraldeli S P, Carmo A R P, Sato C T, Rosa B T. Evaluation of dentin bond strengths: In vitro vs. in vivo. J Dent Res 2002; 80(437-abstract 375).
14. Geraldeli S P, Larson W K. A novel micro-tensile dentin bond strength method using paired dentin quarters. J Dent Res 2002; 80(1065):abstract 3373.
15. Belli W A, Marquis R E. Adaptation of Streptococcus mutans and Enterococcus hirae to acid stress in continuous culture. Appl Environ Microbiol 1991; 57(4):1134-8.
16. Giachetti L, Bertini F, Bambi C, Scaminaci Russo D. A rational use of dental materials in posterior direct resin restorations in order to control polymerization shrinkage stress. Minerva Stomatol 2007; 56(3):129-38.
17. Mullejans R, Lang H, Schuler N, Baldawi M O, Raab W H. Increment technique for extended Class V restorations: an experimental study. Oper Dent 2003; 28(4):352-6

1. A restorative dental material comprising:
a restorative dental composition and arginine incorporated into the restorative dental material such that, in the oral cavity of a host, arginine is released from the restorative dental material over time.

2. The restorative dental material of claim 1, wherein the restorative dental material is permanent or semi-permanent.

3. The restorative dental material of claim 1, wherein the restorative dental composition comprises a polymer and the arginine forms particles within the polymerized dental composition.

4. The restorative dental material of claim 1, wherein the arginine comprises about 2-15% by weight of the restorative dental material.

5. The restorative dental material of claim 1, wherein the arginine comprises about 5-7% by weight of the restorative dental material.

6. The restorative dental material of claim 1, wherein the arginine is released from the restorative dental material into the oral cavity of a host at a rate of about 0.2 to about 75.0 μmol/cm².

7. The restorative dental material of claim 1, wherein the restorative dental composition is made from a restorative dental composition combination, pre-mix, or kit, wherein the combination, pre-mix, or kit comprises ingredients for making a restorative dental composition, and an amount of arginine is combined with the combination, pre-mix, or kit of ingredients.

8. The restorative dental material of claim 1, wherein the restorative dental material is selected from the group of materials consisting of: dental bonding materials, dental adhesives, resin cements, resin composites, sealants, varnishes, resin modified glass ionomers, prosthodontic and orthodontic appliances, customized trays for dental purposes.

9. The restorative dental material of claim 1, wherein the restorative dental material comprises a dental adhesive.

10. The restorative dental material of claim 9, wherein the dental adhesive comprises monomers selected from methacrylate monomers, acrylate monomers and combinations thereof, wherein the monomers polymerize to form an adhesive bond.

11. The restorative dental material of claim 10, wherein the dental adhesive further comprises a filler material.

12. The restorative dental material of claim 11, wherein the filler is selected from the group consisting of: silanized glass, amorphous and/or colloidal silica, polyacrylic acid polymers, ceramics, quartz, organically modified ceramic, and combinations thereof.

13. The restorative dental material of claim 11, wherein the filler comprises silanized glass.

14. The restorative dental material of claim 10, wherein the arginine comprises about 2-15% by weight of the restorative dental material.

15. A dental adhesive composition for use in restorative dentistry comprising:
a polymerizable compound or mixture of compounds capable of polymerizing to form an adhesive bond;
an optional filler material; and
arginine, wherein the arginine is released from the dental adhesive composition into an oral cavity of a host over time.

16. The dental adhesive composition of claim 15, wherein the polymerizable compound is selected from the group consisting of: methacrylate monomers, acrylate monomers and combinations thereof.

17. (canceled)

18. (canceled)

19. A method of reducing growth, metabolic activity, or both, of a caries-inducing pathogen associated with restorative dental materials, the method comprising:
providing a restorative dental material comprising arginine, and
placing the restorative dental material in the oral cavity of a host, wherein the arginine is released from the restorative dental material into the oral cavity of the host over time and wherein the arginine release is sufficient to reduce the growth, metabolic activity, or both, of a caries-inducing pathogen in the area of the oral cavity adjacent to the restorative dental material.

20. The method of claim 19, wherein the caries associated with restorative dental materials comprises carries around the margins of a dental filling.

21. The method of claim 19, wherein the arginine in the restorative dental material increases the pH of oral biofilms located adjacent to the restorative dental material.

22. (canceled)

23. The method of claim 22, wherein the caries-inducing pathogen is Streptococcus mutans.

24-25. (canceled)
